(19) United States
(12) Plant Patent Application Publication
(10) Pub. No.: US 2022/0369528 P1
(43) Pub. Date: Nov. 17, 2022

(54) Sedum plant named 'Spellbound'

(50) ;

(71) Applicant: Hans A. Hansen, Zeeland, MI (US)

(72) Inventor: Hans A. Hansen, Zeeland, MI (US)

(73) Assignee: Walters Gardens Inc, Zeeland, MI (US)

(21) Appl. No.: 17/300,288

(22) Filed: May 12, 2021

(51) Int. Cl. A01H 6/32

(52) U.S. Cl. PLT/479

(57) ABSTRACT

The new and distinct cultivar of perennial Sedum plant named ‘Spellbound’ is characterized by tight-forming compact clumps with greyed-green to greyed-purple glaucous foliage on vertical to slightly highly branching flower stems that resist falling over and opening up in the center of the plant throughout the growing season. Numerous stems produce a multitude of flowers beginning as soft-pink buds and opening to creamy-colored flowers that completely cover the top and sides of the domed plant. The new plant is useful for the landscape in containers, as specimen or en masse.

Botanical designation: Sedum hybrid.

Cultivar denomination: ‘Spellbound’.

### STATEMENT REGARDING PRIOR DISCLOSURES UNDER 37 CFR 1.77(b)(6)

The claimed plant was first sold privately to Swift Greenhouses, Inc. on May 15, 2020 by Ball Horticultural., who obtained the plant and all information relating thereto, indirectly from the inventor. Subsequently the new plant was also sold privately to Prides Corner Farms on Jun. 20, 2020 by Walters Gardens, Inc. who also obtained the new plant and all information relating to it from the inventor. No plants of Sedum ‘Spellbound’ have been sold in this country or anywhere in the world, nor has any disclosure of the new plant been made, more than one year prior to the filing date of this application, and such sale or disclosure within one year was either derived directly or indirectly from the inventor.

### BACKGROUND OF THE INVENTION

The present invention relates to a new and distinct cultivar of Sedum plant, botanically known as Sedum and hereinafter referred to by the cultivar name ‘Spellbound’. There are over 300 recognized species of the genus Sedum.

The new Sedum plant is a selection from a planned breeding program conducted by the inventor, at a wholesale perennial nursery in Zeeland, Mich., USA.

The new Sedum plant was selected as a single seedling from a self-pollinated cross in late summer of 2014 of a proprietary hybrid known only as 12-50-16 (not patented). Sedum ‘Spellbound’ was assigned the breeder code number 14-23-17 to keep it separated from among thousands of other seedlings of this and other crosses when it progressed through the initial trial phase in the fall of 2016. The selected single seedling clone was originally selected for it strong, healthy, resistance to lodging, upright growth, colorful foliage and good flower coverage.

Asexual reproduction of Sedum ‘Spellbound’ by vegetative shoot tip cuttings at a wholesale perennial nursery in Zeeland, Mich., USA since the spring of 2017, has shown that the unique features of this new Sedum plant are stable and reproduced true to type in successive generations.

No plants of Sedum ‘Spellbound’ have been sold or disclosed by this or any other name, in this country or anywhere in the world, with the exception of that which was either derived directly or indirectly from the inventor and disclosed within one year of the filing of this application.

### SUMMARY OF THE INVENTION

Plants of Sedum ‘Spellbound’ have not been observed under all possible environmental conditions. The phenotype may vary somewhat with variations in environment such as temperature, available water, fertility and light intensity without, however, any variation in genotype.

The following traits have been repeatedly observed and are determined to comprise the unique characteristics of Sedum ‘Spellbound’ and distinguish it as a new and distinct autumn stonecrop plant unique from all other autumn stonecrop plants known to the inventor:
1. Tight-forming compact clumps with vertical highly branched stems.
2. Center stems remain erect throughout the growing season to maintain full habit.
3. Rounded, greyed-green to greyed-purple, glaucous foliage.
4. Numerous, branched, greyed-purple stems produce soft-pink flower buds opening to creamy-colored flowers;
5. Flowers nearly completely cover the whole domed plant in peak bloom.

In comparison with the parent, the parent has foliage that is more grey-purple colored rounded, fleshy and greyed-purple colored foliage; the inflorescences are more purplish red; and the habit is less stiff and less upright.

The nearest comparison varieties known to the inventor are ‘Night Embers’ U.S. Plant Pat. No. 29,211, ‘Matrona’ (not patented) and ‘Maestro’ U.S. Plant Pat. No. 20,094. ‘Night Embers’ is taller and more upright in habit, with fewer stems of dark mahogany and flowers of mauve pink with less coverage of the top of the plant. ‘Matrona’ is slightly taller in habit with less branching and the flower color is light pink with a deeper pink gynoecium. ‘Maestro’ is slightly taller in habit with less branching, darker mahogany foliage and flowers of rosy pink.

### BRIEF DESCRIPTION OF THE PHOTOGRAPHS

The photograph of the new plant demonstrates the unique traits and the overall appearance of Sedum ‘Spellbound’. The colors are as accurate as reasonably possible with color reproductions of this type. Variations in ambient light spectrum, source and direction may cause the appearance of minor variation in color. The plants used in the photograph were one and three-year-old plants grown in an open, full-sun trial garden at a wholesale perennial nursery in Zeeland, Mich. with supplemental water and fertilizer when needed. No plant growth regulators or pinching have been used.

FIG. 1 shows the plant in early stages of flowering with foliage showing.

FIG. 2 shows the plant in full flower.

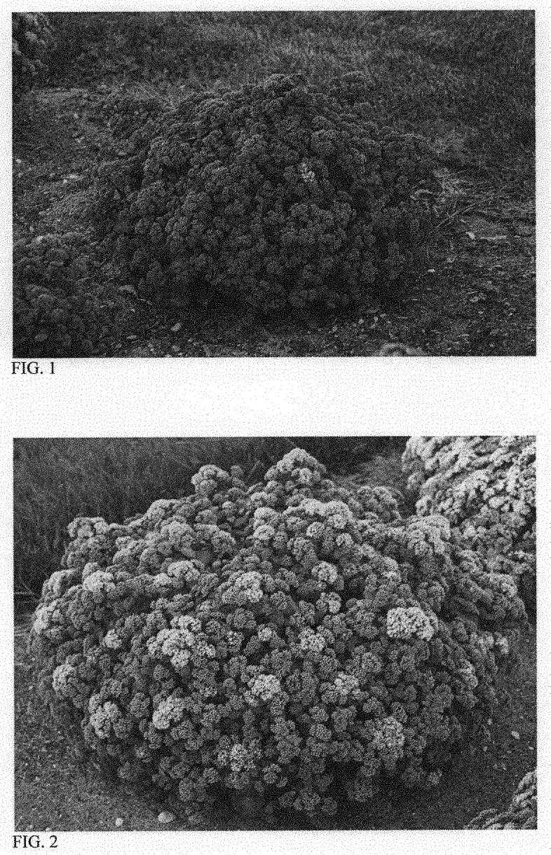

### DETAILED BOTANICAL DESCRIPTION

The following is a detailed description of three-year-old plants of the new cultivar as grown outdoors in a full sun trial plot at a wholesale nursery in Zeeland, Mich. No plant growth regulators or pinching have been used. Plants of the new cultivar have not been tested under all possible conditions. The phenotype may vary with changes in environment, climate, and cultural conditions without change however in the genotype. The color reference is in accordance with the 2015 edition of The Royal Horticultural Society Colour Chart except where general color dictionary terms are used.
Botanical designation: Sedum hybrid;
Cultivar denomination: ‘Spellbound’;
Parentage: Self-pollination of the proprietary hybrid 12-50-16;
Asexual propagation: Stem tip cuttings and division;
Time to initiate roots: About two weeks;
Time to finish a 3.8 liter flowering container: About two months in the summer from a rooted 2.5 cm plug;
Root description: Thick, fleshy; freely branching; creamy white to light tan in color.
Plant habit: Winter-hardy herbaceous perennial; clumping mound; stems stiff and upright to arching arising from a dense crown; dense and full, not opening in center later in the season;
Growth rate: Moderately vigorous;
Plant size: About 40.0 cm tall and 66.0 cm wide in full flower;
Stems: Terete, glaucous, glabrous; about 25.0 cm long from base to initial branches and diameter at base average about 8.0 mm; internodes average about 8.0 mm; about 28 stems per plant;
Stem color: Young nearest a blend between RHS N138D and RHS 191B, mature between RHS 146D and RHS 145A with strong blushing to solid nearest RHS 59A;
Lateral branches: Glaucous; branches about 24.0 cm long and 5.0 mm diameter;
Lateral branch color: Between RHS 146D and RHS 145A with strong blushing to solid nearest RHS 59A;
Foliage: Obovate to orbicular; opposite to sub-opposite; simple; smooth, glabrous, glaucous on both surfaces; sessile; thick, fleshy and succulent; apex rounded; base cuneate to rounded; margin irregularly and shallowly dentate, about 3.0 mm to 9.0 mm apart; decreasing in size distally; attitude outward; fragrancy not observed;
Foliage size: To about 4.5 cm long, 3.5 cm across and 3.0 mm thick; average about 3.3 cm long, 2.2 cm across and 2.5 mm thick;
Foliage color: Young adaxial nearest RHS N138B, young abaxial nearest RHS 148B lightly speculate with nearest RHS N186C; mature adaxial nearest RHS 191A with light blush of nearest RHS N187A, mature abaxial nearest RHS 191B with faint blush of nearest RHS N187B;
Petiole: Sessile;
Venation: Palmate, barely distinguishable; slightly raised abaxial;
Vein color: Adaxial midrib nearest RHS 139D; abaxial midrib nearest RHS 138b; adaxial and abaxial veins same as surrounding leaf tissue;
Flower: Stellate, actinomophic; pentamerous; persistent; attitude upright to outwardly in terminal compound cymes; about 7.0 mm across and 6.0 mm deep;
Flower number: About 3,000 to 5,000 flowers per plant;
Fragrance: None detected;
Flowering season: Beginning late summer to early fall in Zeeland, Mich.;
Longevity: Flower cymes remain effective for about three weeks on the plant and one week as cut flowers; individual flowers persistent and effective for about one week;
Flower buds about one day prior to opening: Broadly ellipsoidal with five angular sides, semi-carinate; apex acute; base acute; about 3.0 mm diameter and about 4.0 mm long;
Flower bud color about one day prior to opening: Blend between RHS 31D and RHS N170D; color of buds about ten days prior to opening blend between RHS 186C and RES 186D;
Inflorescence: Flowering in the upper 6.5 cm and about 10.0 cm across; up to about 150 flowers per inflorescence;
Petals: Typically five, broadly-lanceolate; acute apex, base fused, margin entire; glabrous and lustrous abaxial and adaxial; average about 5.5 mm long and about 2.5 mm across at fusion;
Petal color: Adaxial nearest RHS 11D at anthesis lightening to nearest RHS NN155B in maturity; abaxial nearest RHS NN155B;
Calyx: With five sepals, fused at base; adpressed to petals, about 3.0 mm long and about 5.0 mm diameter;
Sepals: Campanulate; lanceolate; acute apex; fused base; margin entire; glabrous and glaucous both abaxial and adaxial; about 2.5 mm long and about 2.5 mm wide at fused base;
Sepal color: Abaxial and adaxial nearest a blend between RHS N187C and RHS N170D;
Peduncles: Terete; slightly glaucous, glabrous; stiff and flexible; freely branching; upright to slightly outwardly; with branches to about 40 degrees from perpendicular; to about 24.0 cm long and 7.0 mm diameter;
Peduncle color: Between RHS 146D and RHS 145A with strong blushing to solid nearest RHS 59A;
Pedicels: Terete; glabrous, slightly glaucous; strong and stiff, yet flexible; about 4.5 mm long and 1.0 mm diameter;
Pedicel color: Blend between RHS N187B and RHS N186C;
Androecium: Typically ten stamens;
   Filaments.—Outwardly; about 6.0 mm long and about 0.2 mm diameter; color between RHS 1D and RHS NN155B;
   Anthers.—Basifixed, longitudinal; globose to ellipsoid; about 1.0 mm long and 0.6 mm diameter; color nearest RHS N77D;
   Pollen.—Moderate in quantity; color nearest RHS 18C;
Gynoecium: Typically five; ellipsoidal proximally and conic in distal one third; about 4.5 mm long and 1.5 mm diameter;
   Style.—Terete; about 1.0 mm long and 0.5 mm diameter at point of attachment to ovary; glabrous; color at anthesis nearest RHS 3C, becoming nearest RHS NN155B on old flowers;
   Stigma.—Minute, globose; about 0.2 mm diameter and 0.2 mm long; color nearest RHS NN155B;
   Ovary.—Ellipsoidal, nearly terete, acutely tapering at apex to style; base truncate; lustrous; about 4.0 mm long and 1.5 mm diameter; color at anthesis between RHS 3C, and nearest RHS 24D after pollen dehisced;
Fruit: Ventrally dehiscent follicle; about 3.5 mm long and 3.2 mm across; color at maturity nearest RHS 177A;
Seed: Sparse; fine; lanceolate; about 1.0 mm long and less than 0.2 mm across; color between RHS 165A and RHS 165B;
Disease and pest resistance: Plants of the new Sedum ‘Spellbound’ is xeromorphic and grow best with good drainage, full sun and moderate to low fertility. ‘Spellbound’ is cold hardy from USDA zones 3 to 9 and has tolerated temperatures of at least 35 degrees C. ‘Spellbound’ tolerates heavy rains and wind and is not prone to develop an open center later in the season as many other Sedum cultivars do. The new plant is resistant to powdery mildew. Other pest and disease resistance and tolerance outside that normal for Sedum is not known.

It is claimed:

1. The new and distinct perennial Sedum plant named ‘Spellbound’ as herein described and illustrated.
